(19) United States
(12) Patent Application Publication
(10) Pub. No.: US 2012/0277861 A1
(43) Pub. Date: Nov. 1, 2012

(54) EXPANDABLE SPINAL INTERBODY IMPLANT

(75) Inventors: Bradley Steele, Germantown, TN (US); Matthew J. Van Nortwick, Germantown, TN (US)

(73) Assignee: WARSAW ORTHOPEDIC, INC., Warsaw, IN (US)

(21) Appl. No.: 13/096,668

(22) Filed: Apr. 28, 2011

(51) Int. Cl. A61F 2/44

(52) U.S. Cl. 623/17.12; 623/17.16

(57) ABSTRACT

An expandable spinal implant for insertion into and positioning in an intervertebral disc space for intervertebral stabilization, the implant comprising a first implant section, an opposing second implant section and at least one expander attached between the first and second implant sections. The at least one implant expander can be selectively expanded to thereby actuate the spinal implant from a collapsed to an expanded position. The at least one implant expander is expanded via injection of an expanding material, such as bone cement. Also, the at least one implant expander is selectively expanded to impart substantially vertical expansion, angular expansion or vertical and angular expansion to the spinal implant. The expandable implant comprises polyetheretherketone (PEEK) or Titanium implant sections coupled to the implant expander whereby the implant is positioned between adjacent vertebral endplates where the implant can be expanded in the disc space between adjacent vertebral bodies.

### BACKGROUND

The present application is directed to implants, devices and methods for stabilizing vertebral members, and more particularly, to intervertebral implants, devices and methods of use in replacing, in whole or in part, an intervertebral disc, a vertebral member, or a combination of both to distract and/or stabilize the spine.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions and ailments may lead to damage of the spine, intervertebral discs and/or the vertebral members. The damage may result from a variety of causes including, but not limited to, events such as trauma, a degenerative condition, a tumor, or infection. Damage to the intervertebral discs and vertebral members can lead to pain, neurological deficit, and/or loss of motion of the spinal elements.

Various procedures include replacing a section of or an entire intervertebral disc, a section of or an entire vertebral member, or both. One or more spinal implants may be inserted to replace damaged discs and/or vertebral members. The implants are configured to be inserted into an intervertebral space and contact against adjacent vertebral members. The implants are intended to reduce or eliminate the pain and neurological deficit, and increase the range of motion.

The curvature of the spine and general shapes of the vertebral members may make it difficult for the implants to adequately contact the adjacent vertebral members or to position the adjacent vertebral members in a desired orientation. There is a need for spinal implants or devices configurable to match the spinal anatomy for secure contact and/or desired orientation of the spinal implants or devices implanted into an intervertebral disc space.

### SUMMARY

The present application discloses a spinal implant for insertion into and positioning in an intervertebral disc space. The implant comprises a first implant section, an opposing second implant section and at least one expander attached between the first and second implant sections. The at least one implant expander can be selectively expanded to thereby actuate the spinal implant to an expanded position. The at least one implant expander is expanded via injection of an expanding material. The at least one implant expander is selectively expanded to impart substantially vertical expansion, angular expansion or vertical and angular expansion to the spinal implant. The at least one expander can be a balloon that accepts bone cement as the expanding material. The at least one implant expander can impart vertical expansion in the range of between eight and fourteen millimeters (8-14 mm) or an angular expansion in the range of between zero degrees to twelve degrees (0°-12°). The first and second implant sections can be polyetheretherketone (PEEK) or a metallic material such as titanium (Ti).

The present application also discloses a biocompatible expandable spinal implant for insertion into an intervertebral space between adjacent vertebral members. The expandable implant imparts, distracts and restores desired disc space height and angular orientation to adjacent vertebral bodies when the implant is positioned and expanded in the intervertebral disc space and enables fusion of the adjacent vertebrae. The implant comprises a first implant section comprising bone securing serrations, an opposing second implant section comprising bone securing serrations, a first lateral expander attached between the first and second implant sections, and an opposing second lateral expander attached between the first and second implant sections. The first and second lateral expanders can be selectively expanded to actuate the spinal implant to an expanded position. The first and second lateral expanders can be expanded via injection of an expanding material. The first and second lateral expanders can be selectively expanded to impart substantially vertical expansion, angular expansion or vertical and angular expansion to the spinal implant. The first and second lateral expanders can be a balloon that accepts bone cement as the expanding material. The first and second lateral expanders can impart vertical expansion in the range of between eight and fourteen millimeters (8-14 mm) or an angular expansion in the range of between zero degrees to twelve degrees (0°-12°). The first and second lateral expanders can be polyetheretherketone (PEEK) or a metallic material such as titanium (Ti).

The various aspects of the various embodiments may be used alone or in any combination, as is desired. Disclosed aspects or embodiments are discussed and depicted in the attached drawings and the description provided below.

### BRIEF DESCRIPTION OF THE DRAWINGS

FIG. 1 is sagittal plane view of an implant according to one embodiment of the present disclosure positioned in an intervertebral space between vertebral members;

FIG. 2 is a perspective view of an implant in a collapsed position according to one embodiment of the present disclosure;

FIG. 3 is a perspective view of the implant of FIG. 2 in an expanded position;

FIG. 4A is a side view of the spinal implant of FIG. 3;

FIG. 4B is a rear view of the spinal implant of FIG. 3;

FIG. 5A is a partial perspective view of the implant of FIG. 3;

FIG. 5B is a perspective view of the implant of FIG. 3 along section line A-A;

FIG. 5C is a perspective view of the implant of FIG. 3 along section line B-B;

FIG. 6A is a rear view of an implant in a collapsed position according to a second embodiment of the present disclosure;

FIG. 6B is a rear view of the implant of FIG. 6A in an expanded position;

FIG. 7A is a rear view of an implant in a collapsed position according to a third embodiment of the present disclosure; and

FIG. 7B is a rear view of the implant of FIG. 7A in an expanded lordotic position.

### DETAILED DESCRIPTION

The present disclosure is directed to intervertebral implants for spacing apart vertebral members. The present disclosure relates to medical devices such as spinal intervertebral implants implanted between adjacent vertebral bodies of a spinal column section, and methods of use. More particularly, to an expandable spinal implant with opposing upper and lower implant sections coupled to one or more expandable component or expander such as a bag, balloon, pouch, or sac, where the opposing upper and lower implant sections include surface serrations, teeth, texture or extensions enable the assembled expandable spinal implant to be securely positioned between adjacent vertebral endplates. The expandable implant imparts, distracts and restores desired disc space height in adjacent vertebral bodies when the implant is positioned in the intervertebral disc space. The disclosed expandable spinal implant comprises properties which enable the expandable spinal implant to be expanded vertically to increase the implant's overall height, lordotically to angularly expand the spinal implant or a combination of height and lordotic implant expansion. For purposes of promoting an understanding of the principles of the invention, reference will now be made to one or more embodiments or aspects, examples, drawing illustrations, and specific language will be used to describe the same. It will nevertheless be understood that the various described embodiments or aspects are only exemplary in nature and no limitation of the scope of the invention is thereby intended. Any alterations and further modifications in the described embodiments or aspects, and any further applications of the principles of the invention as described herein are contemplated as would normally occur to one skilled in the art to which the invention relates.

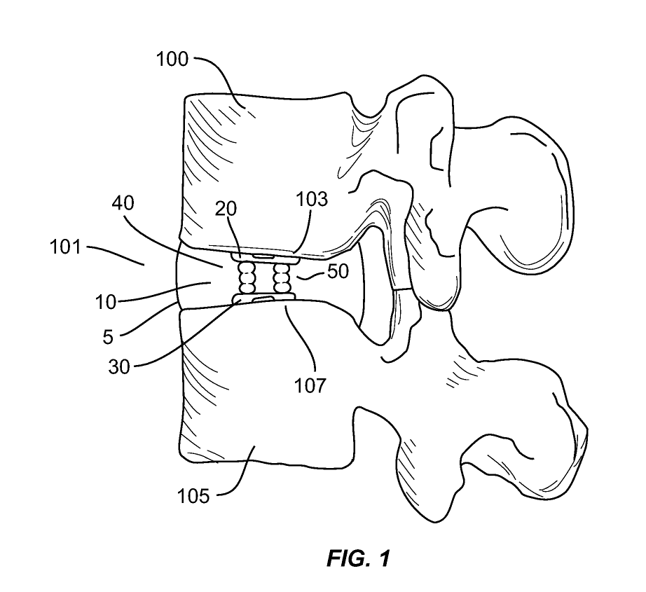

FIG. 1 illustrates a sagittal plane view of vertebral joint section or motion segment of a vertebral column. A spinal implant or device 10 is positioned in an intervertebral disc space 101 between adjacent vertebral members 100 and 105. The upper and lower vertebral bodies 100 and 105 include respective end plates 103 and 107. An intervertebral disc space 101 is located between the endplates 103 and 107. An intervertebral disc 5 is located in the intervertebral disc space 101 between the adjacent endplates 103 and 107 and around the periphery of the disc space 101. The intervertebral disc 5 is comprised of an annulus fibrosus or annulus which surrounds a nucleus pulposus. FIG. 1 further depicts an expandable spinal implant, spacer or device 10 comprising opposing upper and lower implant sections 20 and 30 coupled to at least one expandable component or expander 40, positioned in the intervertebral disc space 101. The spinal implant 10 can be used to promote fusion or preserve motion between adjacent vertebral bodies 100 and 105, depending on the specific shape or configuration of the implant used in a surgical procedure.

FIG. 1 depicts an implantation technique where the spinal implant 10 has been delivered to the intervertebral disc space 101, for example via a known surgical technique such as a direct lateral lumbar interbody fusion (DLIF) approach and procedure. Such a spinal implant DLIF procedure and approach is a well known surgical implant procedure and delivery approach for delivery and insertion of a spinal implant 10 into a desired or selected intervertebral disc space 101. Those of skill in the art will recognize that the spinal implant 10 could also be delivered and inserted in the disc space 101 so as to have different orientations and positions in the disc space 101 between the adjacent vertebrae 100 and 105. For example using known surgical approaches, including, anterior, posterior, direct lateral, translateral, posterolateral, anterolateral or any other suitable oblique direction desired or required by a surgeon or medical application. The spinal implant 10 could also be delivered and inserted in the disc space 101 using other well known surgical procedures and techniques, including among others, anterior lumbar interbody fusion (ALIF), posterior lumbar interbody fusion (PLIF), transforaminal lumbar interbody fusion (TLIF) or other known surgical procedures or techniques desired or required by a surgeon or medical application. Further, those of skill in the art will also recognize that a spinal implant 10 may be delivered and inserted through known surgical techniques and procedures via open, mini-open, minimal access spinal technologies (MAST) or other minimally invasive surgical (MIS) techniques. Moreover, delivery and insertion of the present spinal implant 10 is contemplated through the use of typical and existing instruments presently known and used in existing surgical approached, procedures and techniques.

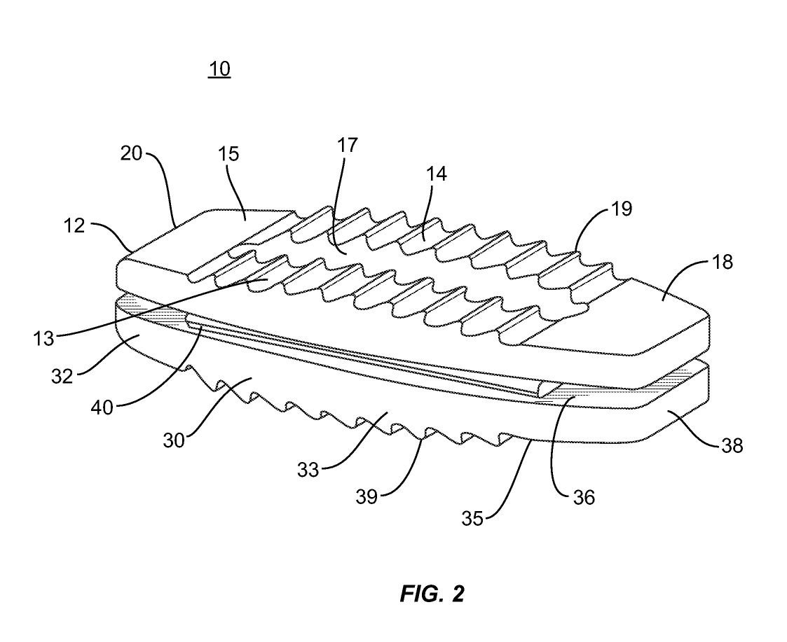

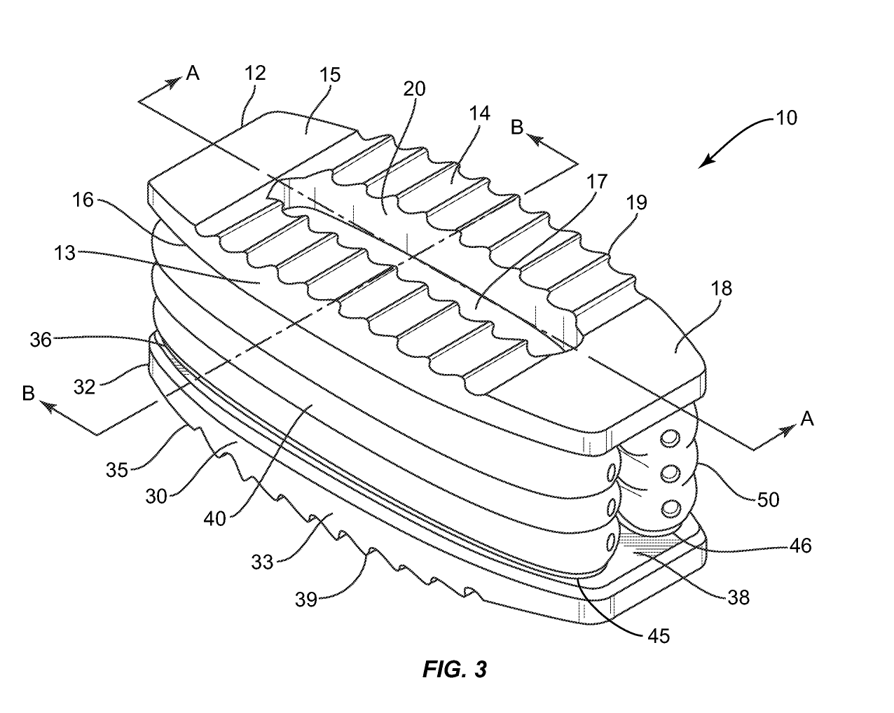

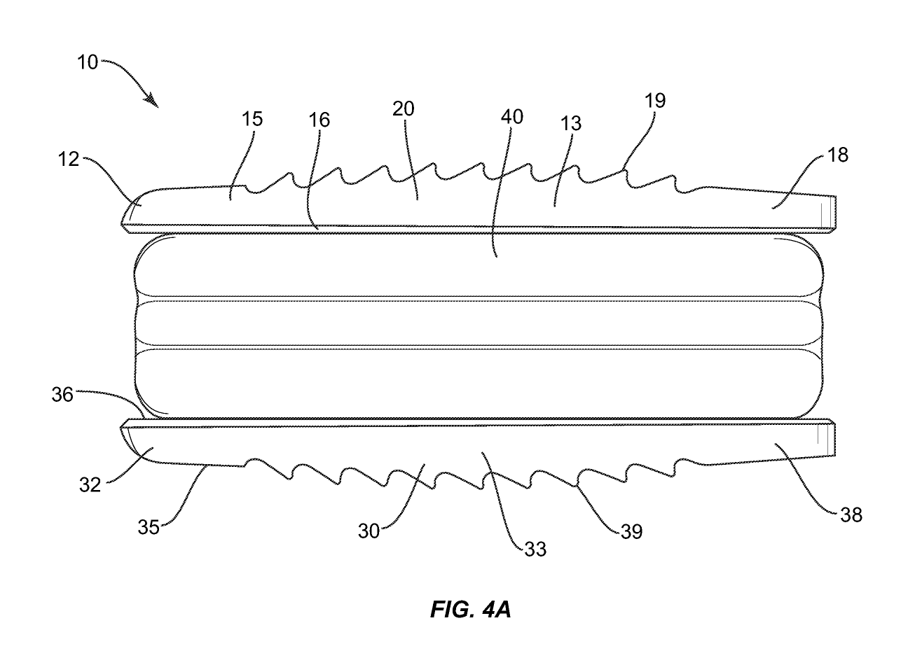

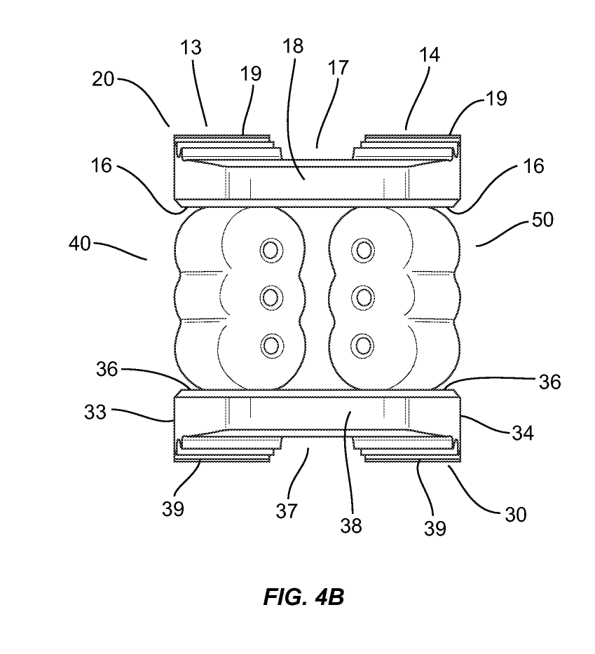

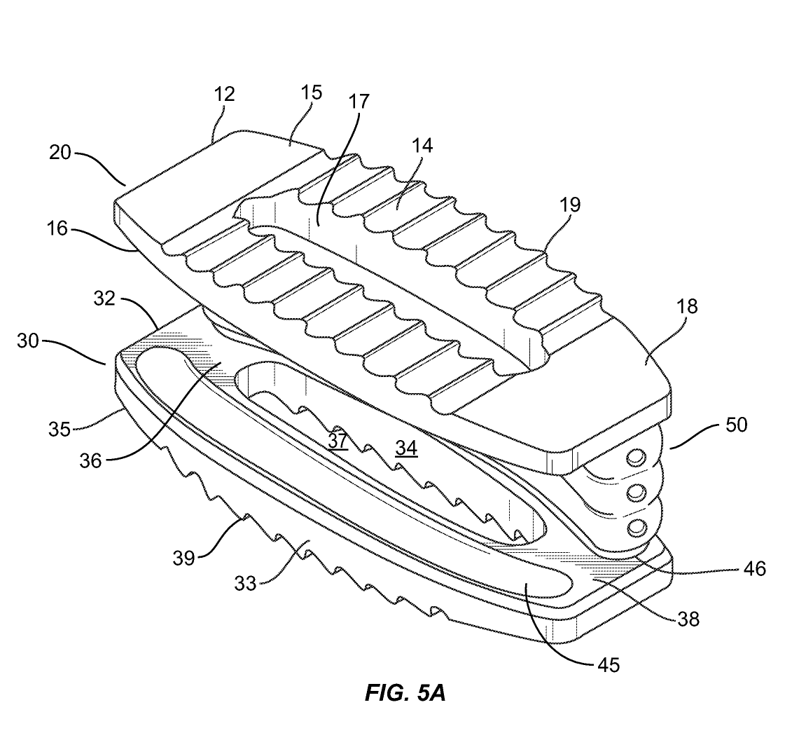

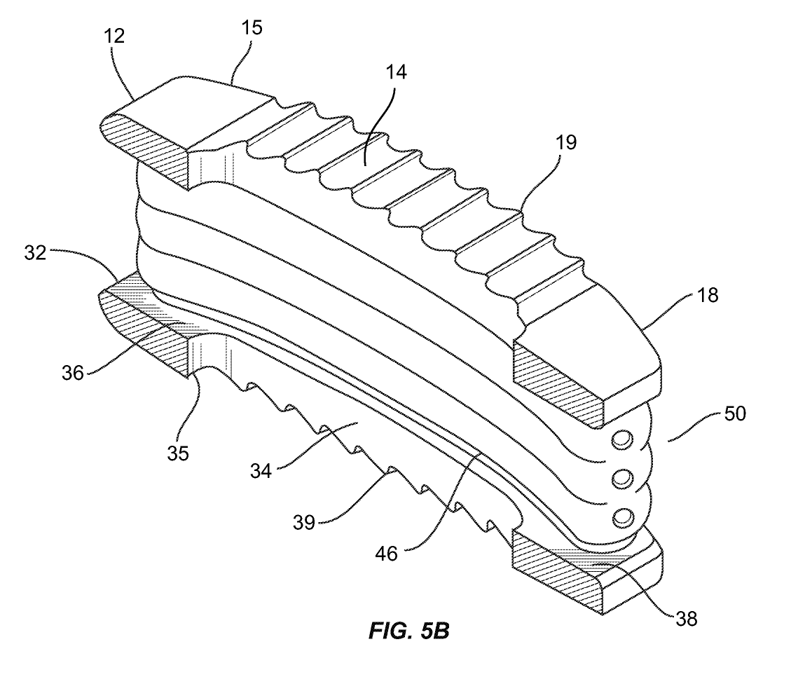

FIGS. 2-5C illustrate a spinal implant 10 according to a preferred aspect of the present disclosure. FIG. 2 is a perspective view of an expandable spinal implant 10 in a collapsed position according to one embodiment of the present disclosure. FIG. 3 is a perspective view of the implant 10 of FIG. 2. FIGS. 4A and 4B are side and rear views of the expandable spinal implant of FIG. 3. FIG. 5A is a partial perspective view of the expandable spinal implant 10 of FIG. 3. FIGS. 5B and 5C are perspective views of the expandable implant of FIG. 3 along section lines A-A and B-B, respectively. In a preferred aspect, the expandable spinal implant 10 comprises an upper implant section 20, an opposing lower implant section 30 and first and second expandable components or expanders 40 and 50. The first expandable component or expander 40 is preferably secured or attached between the upper implant section 20 and a lower implant section 30. An opposing second expandable component or expander 50 is also secured or attached between the upper implant section 20 and a lower implant section 30. The expandable implant 10 can take on or transition between a collapsed position, as shown in FIG. 2, and an expanded position, as shown in FIG. 3.

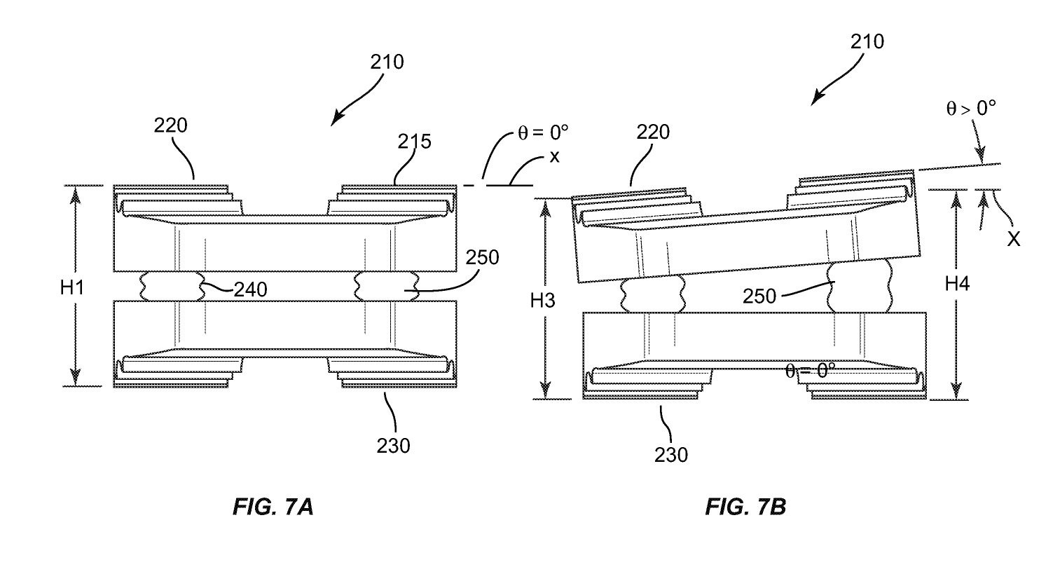

Additionally, the expandable spinal implant 10 can be selectively controlled to expand such that the expandable spinal implant 10 can be expanded vertically to increase the implant's overall height by expanding the first and second expandable or inflatable components or expanders 40 and 50 by the same amount, as shown in FIGS. 2, 3 and 6A-6B. The expandable spinal implant 10 can also be selectively controlled to expand such that the expandable spinal implant 10 expands to have a lordotic or angular configuration by expanding the first and second expandable or inflatable components or expanders 40 and 50 by a different amount, as shown in FIGS. 7A-7B. Those of skill in the art will recognize that the expandable spinal implant 10 can also be selectively controlled to expand such that the expandable spinal implant 10 can be expanded both vertically to increase the implant's overall height and lordotically to result in a lordotic or angular configuration by selectively varying the amount of expansion of the first and second expandable components or expanders 40 and 50. In this manner, the implant 10 can impart a combination of height and lordotic implant expansion as may be needed by a surgeon or an implant procedure or application.

In the preferred aspect shown in FIGS. 2-5C, there are two identical first and second expandable components or expanders 40 and 50 which are respectively coupled or attached between the upper implant section 20 and lower implant section 30. However, those of skill in the art will recognize that the first and second expandable components or expanders 40 and 50 may also have or take on a different sizes and configurations as may be needed by a surgeon or a spinal implant procedure or application. Additionally, instead of two identical first and second expandable components or expanders 40 and 50, a spinal implant 10 may instead have a single expandable component or expander (not shown). For example, such an expandable component or expander might have a toroid, ring or other complimentary shaped configuration which is coupled or attached between the upper and lower implant section 20 and 30. In such a case, the single expandable component or expander would also span the implant 10 between the between the upper and lower implant sections 20 and 30 in order to permit the expandable spinal implant 10 to take on or transition between a collapsed position and an expanded position. Whether one or more expandable components or expanders (not shown) are used between the upper and lower implant section 20 and 30 will depend on the selection or requirements of a surgeon or medical procedure or application. Additionally, the expandable spinal implant 10 can comprise an overall shape, configuration or size as may be needed by a surgeon or an implant procedure or application.

In a preferred aspect, the expandable spinal implant 10 comprises an upper implant section 20, an opposing lower implant section 30 and opposing first and second expandable components or expanders 40 and 50. The upper implant section 20 comprises an exterior section surface 15 defined by the upper portions of a leading end 12, first lateral sidewall 13 and second lateral sidewall 14, and a rear end 18. The upper implant section 20 further comprises an opposing underside interior section surface 16 defined by the lower portions of the leading end 12, first lateral sidewall 13 and second lateral sidewall 14, and rear end 18. The exterior section surface 15 is partially curved or rounded at the leading end 12 above the substantially flat underside interior section surface 16. The leading end 12, in conjunction with an opposing and complimentary lower implant section 30 leading end 32, facilitates entry or insertion of a collapsed implant body 10 into the disc space 101 to thereafter enable selective or desired distraction of collapsed or semi-collapsed adjacent vertebral bodies 100 and 105. The rear end 18, in conjunction with an opposing and complimentary lower implant section 30 rear leading end 38 and first and second expanders 40 and 50, provide a means to attach one or more insertion instruments (not shown) to grasp, attach to and manipulate the insertion, orientation and expansion of the expandable spinal implant 10 as the implant 10 is delivered to a selected or desired disc space 101.

The upper implant section 20 also comprises an upper aperture 17 defined and bounded by the leading end 12, first lateral sidewall 13, second lateral sidewall 14, and rear end 18. The upper implant section aperture 17 is configured such that it is a substantially vertical channel or cavity that extends between and through the exterior and opposing interior section surfaces 15 and 16. In a preferred aspect, the upper aperture 17 permits the insertion of graft material which assists in promoting fusion of the adjacent vertebrae 100 and 105 at the disc space 101 where the implant 10 is inserted. The graft material may be composed of any type of material that has the ability to promote, enhance and/or accelerate the bone growth and fusion or joining together of the vertebral bodies 100 and 105 by one or more fusion mechanisms such as osteogenesis, osteoconduction and/or osteoinduction. The graft material may include allograft material, bone graft, bone marrow, demineralized bone matrix putty or gel and/or any combination thereof. The graft filler material may promote bone growth through and around the upper aperture 17 to promote fusion of the intervertebral joint 100 and 105. Those of skill in the art will recognize that the use of filler graft material is optional, and it may or may not be used depending on the needs or requirements of a physician or a medical procedure.

The exterior section surface 15 of the implant substrate 20 additionally comprises bone securing surface serrations, teeth, projections or extensions 19 which extend outwardly or away from the exterior section surface 15. The exterior bone securing surface serrations or teeth 19 enable the spinal implant 10 to engage adjacent vertebral endplates 103 and 107 so that the implant 10 can be securely positioned between the adjacent vertebral endplates 103 and 107 and act as an anti-ejection mechanism. In the exterior section surface 15 of the implant substrate 20, the bone securing surface serrations or teeth 19 are preferably configured and positioned on the upper portions or sections of the first and second lateral sidewalls 13 and 14, between the upper implant section's leading end 12 and rear end 18. The implant serrations or teeth 19 directly interact with and engage the vertebral endplates 103 and 107 when the spinal implant 10 is positioned in the disc space 101 and provide, in part, stability of the implant 10 in the disc space 101 between adjacent vertebrae 100 and 105.

In the preferred embodiment, the implant serrations or teeth 19 are preferably oriented in a rear lean direction such that the teeth or serrations 19 are oriented away or opposite the upper implant section's leading end 12 and toward the upper implant section's rear end 18. In this manner, the rear leaning orientation of the teeth or serrations 19 provide minimal resistance when the spinal implant 10 is being inserted into a disc space 101. Once inserted, the rear leaning orientation of the teeth or serrations 19 provide a mechanism to prevent the assembled spinal implant 10 from being ejected, or minimize or retard implant movement in a direction tending to eject the implant 10 from the disc space 101, once the spinal implant 10 is positioned in the disc space 101. In the aspects shown in FIGS. 2-7, the teeth or serrations 19 are generally triangular in shape when viewed from a side profile. Those of skill in the art will recognize that the teeth or serrations 19 can instead have other shapes, configurations and sizes including, among others, pyramids, triangles, cones, spikes and keels, as well as different teeth or serration orientation, as may be needed or desired by a physician, procedure or medical application.

The underside interior section surface 16 of the upper implant substrate 20 additionally comprises first and second expander channels 25 and 26, best shown in FIG. 5C, which will complimentarily and cooperatively enable corresponding first and second expandable components or expanders 40 and 50 to be coupled or attached between the upper implant section 20 and a lower implant section 30. As best shown in FIG. 5C, and FIG. 5A for the opposing lower implant section 30, the first and second expander channels 25 and 26 preferably have a substantially concave cross section such that the first and second expander channels 25 and 26 extend inwardly away from the interior section surface 16. The first expander channel 25 extends between the leading end 12 and rear end 18 along the first lateral side wall 13, as best shown in FIG. 5C, and FIG. 5A for an opposing lower implant section 30. The second expander channel 26 extends between the leading end 12 and rear end 18 along the second lateral side wall 14, as best shown in FIG. 5C, and FIG. 5B for an opposing partial lower implant section 30.

The first and second expander channels 25 and 26 will facilitate positioning and secure attachment therein of the respective first and second expandable components or expanders 40 and 50. The secure attachment can be accomplished via a sufficiently and appropriate biocompatible adhesive which will securely bond or attach the first and second expandable components or expanders 40 and 50 to the respective first and second expander channels 25 and 26. In this manner, the upper and lower implant sections 20 and 30 can appropriately translate the force imparted by the expanding first and second expandable components or expanders 40 and 50, as the expandable spinal implant 10 transitioned between a collapsed position, as shown in FIG. 2, and an expanded position, as shown in FIG. 3. Those of skill in the art will readily recognize that other attachments means or mechanisms may be used to securely attach or couple the first and second expander channels 25 and 26 and respective first and second expandable components or expanders 40 and 50. For example, among others, a friction fit, an interference fit, a rough or uneven surface interface, mechanical coupling, thermal bonding or combinations thereof. Those of skill in the art will readily recognize that the first and second expander channels 25 and 26 may have other configurations so long as they are complimentarily and cooperatively enable corresponding first and second expandable components or expanders 40 and 50 to be coupled or attached between the upper and lower implant sections 20 and 30 to permit the expandable spinal implant 10 to transition between a collapsed position, as shown in FIG. 2, and an expanded position, as shown in FIG. 3. The configurations of the first and second expander channels 25 and 26 and corresponding complimentarily first and second expandable components or expanders 40 and 50 will depend on the need or selection of a surgeon or medical procedure or application.

With regard to the lower implant section 30, in the disclosed aspect, shown in FIGS. 2-7, the upper and lower implant sections 20 and 30 have identical configurations and opposing orientations with respect to the first and second expandable components or expanders 40 and 50 positioned therebetween. To that end, the lower implant section 30 exhibits and accomplishes the same but opposing function as the upper implant section 20. The lower implant section 30 comprises an exterior section surface 35 defined by the lower portions of a leading end 32, first lateral sidewall 33 and second lateral sidewall 34, and a rear end 38. The lower implant section 30 also comprises an opposing topside interior section surface 36 defined by the upper portions of the leading end 32, first lateral sidewall 33 and second lateral sidewall 34, and rear end 38. The exterior section surface 35 is partially curved or rounded at the leading end 32 below the substantially flat topside interior section surface 36. The exterior section surface 35 additionally comprises bone securing surface serrations, teeth, projections or extensions 39 which extend outwardly or away from the exterior section surface 35. The lower implant section 30 also comprises a lower aperture 37 defined and bounded by the leading end 32, first lateral sidewall 33, second lateral sidewall 34, and rear end 38.

The topside interior section surface 36 additionally comprises first and second expander channels 45 and 46, best shown in FIGS. 5A-5C, which will complimentarily and cooperatively enable corresponding first and second expandable components or expanders 40 and 50 to be coupled or attached between the upper and lower implant sections 20 and 30. As shown in FIGS. 5A-5C, the first and second expander channels 45 and 46 preferably have a substantially concave cross section such that the first and second expander channels 45 and 46 extend inwardly away from the topside section surface 36. The lower implant section's 30 first expander channel 45 extends between the leading end 32 and rear end 38 along the first lateral side wall 33, as best shown in FIGS. 5A and 5C. The second expander channel 46 extends between the leading end 32 and rear end 38 along the second lateral side wall 34, as best shown in FIGS. 5A-5C. Although, the upper and lower implant sections 20 and 30 have identical configurations and opposing orientations, those of skill in the art, will recognize that the expandable spinal implant 10 may instead use non-identical configurations for the upper and lower implant sections 20 and 30 that permit the expandable components or expanders 40 and 50 to be coupled or attached therebetween to enable the expandable spinal implant 10 to transition between a collapsed position, shown in FIG. 2, and an expanded position, shown in FIG. 3.

In a preferred aspect shown in FIGS. 2-5C, there are two identical first and second expandable components or expanders 40 and 50 which are respectively coupled or attached between the upper and lower implant sections 20 and 30. The first expandable component or expander 40 is preferably positioned between the upper and lower first lateral side walls 13 and 33 and securely attached between the upper and lower first expander channels 25 and 45. The second expandable component or expander 50 is preferably positioned between the upper and lower second lateral side walls 14 and 34 and securely attached between the upper and lower second expander channels 26 and 46. The first and second expandable components or expanders 40 and 50 can be securely attached to respective upper and lower first and second expander channels 25, 26, 45 and 46 via a sufficiently strong adhesive which will securely bond or attach the first and second expandable components or expanders 40 and 50 to the respective upper and lower first and second expander channels 25, 26, 45 and 46. Those of skill in the art will recognize that other attachments means or mechanisms may be used to securely attach or couple the first and second expandable components or expanders 40 and 50 to respective upper and lower first and second expander channels 25, 26, 45 and 46. For example, among others, thermal bonding, a friction fit, an interference fit, a rough or uneven surface interface, mechanical coupling or combinations thereof.

In the preferred aspect, the first and second expandable components or expanders 40 and 50 comprise three individual adjacent and attached tube-like expandable parts or segments 41, 42, 43, 51, 52 and 53. The expandable parts or segments 41, 42, 43, 51, 52 and 53 can be an item such as a balloon, bag, pouch, sac, conduit, duct or other item that can selectively expanded. In one aspect, the expandable balloon, bag, pouch, sac, conduit, duct or other item could be comprised of biocompatible material that degrades or can be absorbed over time such that the injected material and by then hardened material remains as a load bearing structure. Although, three expandable parts or segments 41, 42, 43, 51, 52 and 53 are preferred, more or less expandable parts or segments may be used. For example, the first and second expandable components or expanders 40 and 50 could comprise a single expandable part or segment which is coupled or attached between the upper and lower implant sections 20 and 30. Those of skill in the art will recognize that the number of expandable parts or segments 41, 42, 43, 51, 52 and 53 may vary depend on the need or selection of a surgeon or medical procedure or application. The expandable parts or segments 41, 42, 43, 51, 52 and 53 can be securely attached to each other via a sufficiently strong adhesive which will securely bond or attach adjacent expandable parts or segments 41, 42, 43, 51, 52 and 53 to each other and to respective upper and lower first and second expander channels 25, 26, 45 and 46. Those of skill in the art will recognize that other attachments means or mechanisms may be used to securely attach or couple the adjacent expandable parts or segments 41, 42, 43, 51, 52 and 53 to each other and to respective upper and lower first and second expander channels 25, 26, 45 and 46. For example, among others, thermal bonding, a friction fit, an interference fit, a rough or uneven surface interface, mechanical coupling or combinations thereof.

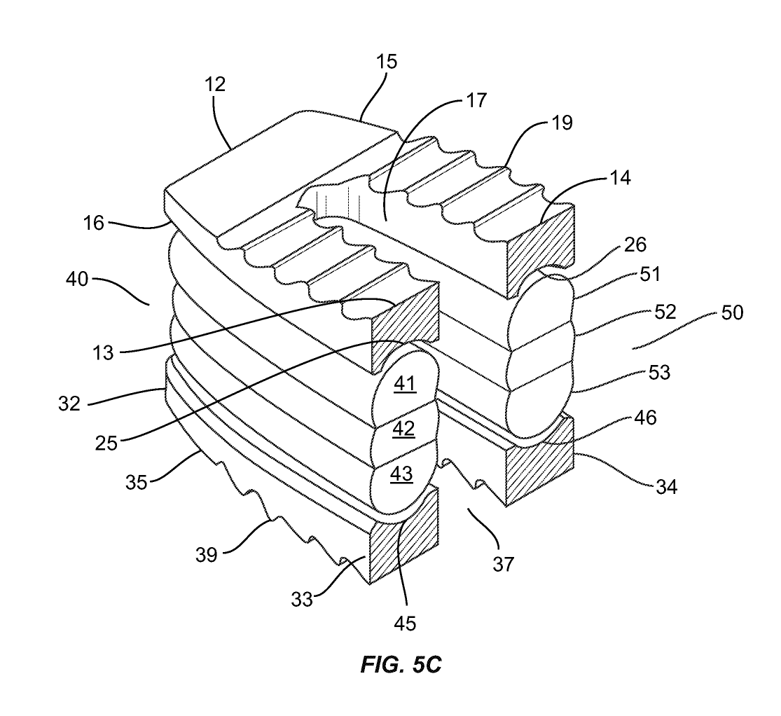

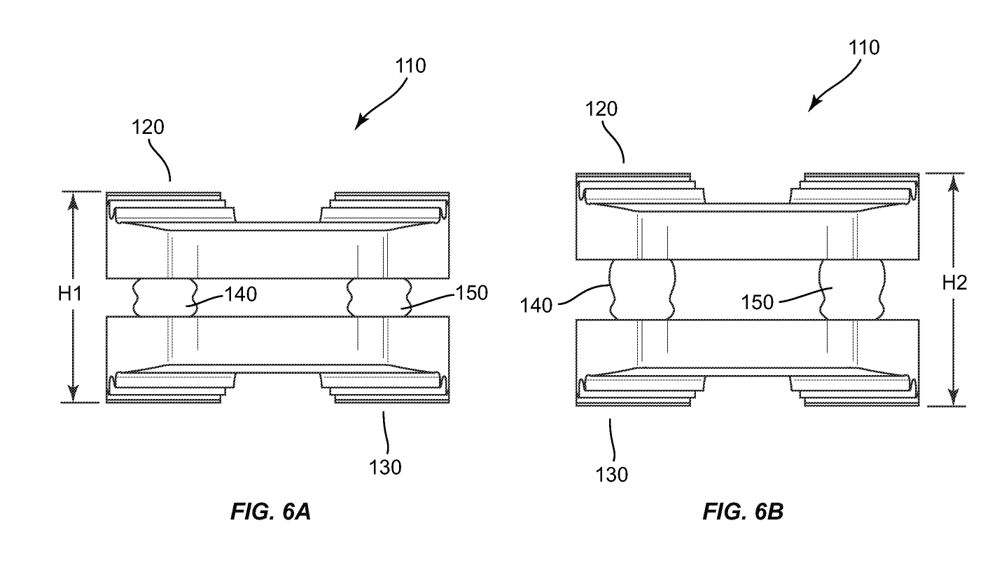

The expandable implant 10 can transition between a collapsed position, shown in FIG. 2, and an expanded position, shown in FIG. 3. Expansion of the first and second expandable components or expanders 40 and 50 enables selective and controlled expansion of the spinal implant 10 in the disc space 101. The expandable spinal implant 10 can be selectively controlled to expand such that the expandable spinal implant 10 can be expanded vertically to increase the implant's overall height, as shown in FIGS. 4A and 6A-6B, lordotically to result in a lordotic or angular configuration, as shown in FIGS. 7A-7B, by selectively varying the amount of expansion of the first and second expandable components or expanders 40 and 50. Those of skill in the art will recognize that the implant 10 could also be selectively expanded to impart a combination of height and lordotic implant expansion as might be needed or required by a surgeon or an implant procedure or application. The selective expansion of the implant 10 may be initiated and carried out by appropriately filling or injecting the individual expandable parts or segments 41, 42, 43, 51, 52 and 53 of the expandable components or expanders 40 and 50 with an appropriate biocompatible solution or material. The filling or injection solution, liquid or material may be, among others, bone cement, ground up allograft, saline solution or any biocompatible material or solution. The controlled delivery of the injection materials into the first and second expandable components or expanders 40 and 50 will result in the selectively and controlled expansion of the spinal implant 10 until the implant 10 reached a desired or needed implant height, lordosis or combination of height and lordosis. The material injected into the expandable components or expanders 40 and 50 is preferably injected so as to fill and expand the individual expandable parts or segments 41, 42, 43, 51, 52 and 53 individually and one at a time. Alternatively, the injected material could be injected into the individual expandable parts or segments 41, 42, 43, 51, 52 and 53 simultaneously. The material injection may be carried out using known instruments and techniques (not shown), and would be injected into the expandable parts or segments 41, 42, 43, 51, 52 and 53 under sufficient pressure to enable the expandable components or expanders 40 and 50 to expand within the disc space 101.

As the expanding material or solution is injected into the parts or segments 41, 42, 43, 51, 52 and 53, the expandable components or expanders 40 and 50 expand, which results in imparting or translating an outward expanding force on the upper and lower implant sections 20 and 30. Continued injection of the material or solution into the parts or segments 41, 42, 43, 51, 52 and 53, and thus continued expansion of the expandable components or expanders 40 and 50, forces the upper and lower implant sections 20 and 30 to continue to expand. As the upper and lower implant sections 20 and 30 continue to expand, they 20 and 30 will in turn impart or translate the outward expanding force to the adjacent vertebral end plates 103 and 107, which will result in expansion of the adjacent vertebrae 100 and 105 and disc space 101. In this manner, the upper and lower implant sections 20 and 30 can appropriately translate the force imparted by the expanding first and second expandable components or expanders 40 and 50, to transition the expandable spinal implant 10 from a collapsed position, as shown in FIG. 2, and an expanded position, as shown in FIG. 3. The expandable spinal implant 10 can thus be selectively expanded to attain, and impart to adjacent vertebrae 100 and 105, an implant height, lordotic configuration or a combination of height and lordosis as may be selected or required by a physician, procedure or medical application.

FIG. 6A is a rear view of an expandable spinal implant 110 in a collapsed position according to a second embodiment of the present disclosure. FIG. 6B is a rear view of the implant 110 of FIG. 6A in an expanded position. In this aspect, the first and second expandable components or expanders 140 and 150 are respectively coupled or attached between the upper and lower implant sections 120 and 130. There first and second expandable components or expanders 140 and 150 will enable the expandable spinal implant 110 to vertically transition between a collapsed position, shown in FIG. 6A, and an expanded position, shown in FIG. 6B. Controlled expansion of the first and second expandable components or expanders 140 and 150 enables selective and controlled vertical expansion of the spinal implant 10 in the disc space 101. The controlled and selective vertical expansion of the implant 110 may be initiated and carried out by injection of biocompatible bone cement, solution or other material into the individual expandable parts or segments of the expandable components or expanders 140 and 150. The first and second expandable components or expanders 140 and 150 can be expanded either sequentially or simultaneously, so long as they both reach the same expanded height H2 as might be needed or required by a surgeon, implant procedure or medical application. The amount of material injected into the first and second expandable components or expanders 140 and 150 will be the same or an appropriate amount so that the first and second expandable components or expanders 140 and 150 both reach the same expanded height H2. In this manner, the implant's overall vertical height can be increased to height H2, as shown in FIGS. 3, 4A and 6B. The expandable spinal implant 110 is controllably expanded to vertically transition from a collapsed position having height H1 to an expanded position H2, where the expanded height H2 is greater than the collapsed height H1, i.e., H2>H1. In one aspect, the expandable spinal implant 110 can have implant heights in the range of 8-14 mm, and its height can increase in 2 mm increments or intervals. As the expandable implant 110 is expanded to the expanded height H2, the upper and lower implant sections 120 and 130 translate the force imparted by the expanding first and second expandable components or expanders 140 and 150 to the adjacent vertebrae 100 and 105. The expandable spinal implant 110 can thereby expand the adjacent vertebrae 100 and 105 and disc space 101 to impart the expanded implant 110 vertical height H2.

FIG. 7A is a rear view of an expandable implant 210 in a collapsed position according to a third embodiment of the present disclosure. FIG. 7B is a rear view of the implant of FIG. 7A in a lordotic expanded position. In this aspect, the collapsed implant 210 shown in FIG. 7A includes a substantially horizontal angular reference line X which is aligned with the top surface 215 of the implant 210 such that the collapsed implant 210 preferably has a lordotic or angular orientation of zero degrees (θ=0°) and a collapsed height of H1. The first and second expandable components or expanders 240 and 250 are respectively coupled or attached between the upper and lower implant sections 220 and 230. There first and second expandable components or expanders 240 and 250 will enable the expandable spinal implant 210 to expand and result in a lordotic or angular transition between a collapsed position, shown in FIG. 7A, and a lordotic expanded position, shown in FIG. 7B. Controlled expansion of the first and second expandable components or expanders 240 and 250 enables selective and controlled lordotic or angular expansion of the spinal implant 210 in the disc space 101. The controlled and selective lordotic expansion of the implant 210 may be initiated and carried out by injection of biocompatible bone cement, solution or other expanding material into the individual expandable parts or segments of the expandable components or expanders 240 and 250. The first and second expandable components or expanders 140 and 150 can be expanded either sequentially or simultaneously, so long as they each reach a different expanded height H3 and H4 which will result in the expandable implant 210 having a selected lordotic or angular orientation (θ>0°) as might be needed or required by a surgeon, implant procedure or medical application. The amount of material injected into the first and second expandable components or expanders 240 and 250 will typically vary so that the expandable implant 210 will reach a selected or desired lordotic or angular orientation θ when the first and second expandable components or expanders 240 and 250 reach different expanded heights H3 and H4.

In this manner, the implant's overall lordotic or angular orientation θ can increase from zero degrees (θ=0°) to a selected or desired lordotic or angular orientation (θ>0°), on a second lateral side with height H4 as shown in FIG. 7B. The expandable spinal implant 210 is controllably expanded to transition from a collapsed position having a lordotic or angular orientation of zero degrees (θ=0°) and height of H1 to an expanded selected or desired lordotic or angular orientation (θ>0°) and a second lateral height H4 greater than a first lateral height H3, i.e., H4>H3. Those of skill in the art will recognize that in an alternate aspect, not shown, the expandable spinal implant 210 could be controllably expanded to transition from a collapsed position to an expanded selected or desired lordotic or angular orientation (θ>0°) on the opposing first lateral side where a first lateral height H3 that is greater than the second lateral height H4, i.e., H3>H4. In one preferred aspect, the expandable spinal implant 110 can be lordotically or angularly expanded to have a lordotic or angular range of orientation of zero to twelve degrees (θ=0°-12°). As the expandable implant 110 is lordotically or angularly expanded to a desired or selected angular orientation θ, the upper and lower implant sections 220 and 230 translate the force imparted by the expanding first and second expandable components or expanders 240 and 250 to the adjacent vertebrae 100 and 105. The expandable spinal implant 210 can thereby lordotically or angularly expand the adjacent vertebrae 100 and 105 and disc space 101 to impart the expanded angular orientation θ and implant 210 heights H3 and H4. Those of skill in the art will recognize that the implant 210 could also be selectively expanded to impart a combination of height and lordotic or angular expansion as might be needed or required by a surgeon or an implant procedure or application. Further, those of skill in the art will recognize that the expandable implant 210 could also be selectively expanded at the implant's leading end and/or rear end to impart selected or desired height, lordotic or angular expansion, or a combination of height and lordotic or angular expansion instead of or in addition to the first and second lateral sides, as might be needed or required by a surgeon or an implant procedure or application.

Additionally, those of skill in the art will recognize that that instead of two identical first and second expandable components or expanders 40, 50, 140, 150, 240 and 250, a spinal implant 10, 110, 210 may instead have a single expandable component or expander (not shown) to impart selected or desired height, lordotic or angular expansion, or a combination of height and lordotic or angular expansion as might be needed or required. For example, such an expandable component or expander might have a toroid, ring or other configuration which is complimentarily coupled or attached between the upper and lower implant section 20, 30, 120, 130, 220 and 230. In such a case, the single expandable component or expander would also span the implant 10 between the between the upper and lower implant sections 20 and 30 in order to permit the expandable spinal implant 10 to take on or transition between a collapsed position and an expanded position. Whether one or more expandable components or expanders (not shown) are used with the upper and lower implant section will depend on the selection or requirements of a surgeon or medical procedure or application. Additionally, the expandable spinal implant can comprise an overall shape, configuration or size as may be needed by a surgeon or an implant procedure or application.

In the disclosed embodiments of FIGS. 2-7B, a spinal implant, commercialized by Medtronic, Inc, under the trademark CLYDESDALE®, is contemplated as using and embodying the advantageous aspects of the spinal implant 10, 110 and 210 disclosed herein. Those of skill in the art will readily recognize that other implant sizes and configuration designs may use or incorporate the advantageous aspects of the spinal implant 10, 110, and 210 disclosed herein. This includes implants having different leading end and rear end configurations. For example, the unique and advantageous aspects of the spinal implant 10, 110 and 210 disclosed herein may be implemented and used in others spinal implants commercialized by a third party, including spinal implants commercialized by Medtronic, Inc, under the trademarks CAPSTONE®, CRESCENT®, etc., along with associated or corresponding delivery and insertion implant instruments. Those of skill in the art will further recognize that implant 10, 110 and 210 could also comprise implant walls which are angled relative to one another. In other embodiments, an implant wall or surface may extend obliquely from an adjacent wall rather than orthogonally. Also, an implant's walls could be tapered, sloped, angled, or curved, including convex, bi-convex and concave curving, depending on a particular medical application need or requirement.

The spinal implants 10, 110, 210 upper and lower implant sections 20, 30, 120, 130, 220 and 230 are preferably comprised of a polyetheretherketone (PEEK) polymer material which allows radiographic assessment of fusion and the bridging bone mass across the disc space while reducing stress-shielding effects. While, the biocompatible upper and lower implant sections 20, 30, 120, 130, 220 and 230 are preferably a radiolucent biocompatible materials such as PEEK, those of skill in the art will recognize that other insert component material may also be used, including among others, carbon fiber reinforced PEEK polymer material, homopolymers, co-polymers and oligomers of polyhydroxy acids, polyesters, polyorthoesters, polyanhydrides, polydioxanone, polydioxanediones, polyesteramides, polyaminoacids, polyamides, polycarbonates, polylactide, polyglycolide, tyrosine-derived polycarbonate, polyanhydride, polyorthoester, polyphosphazene, polyethylene, polyester, polyvinyl alcohol, polyacrylonitrile, polyamide, polytetrafluorethylene, poly-paraphenylene terephthalamide, polyetherketoneketone (PEKK); polyaryletherketones (PAEK), cellulose, carbon fiber reinforced composite, and mixtures thereof. The upper and lower implant sections 20, 30, 120, 130, 220 and 230 may also be comprised of a Titanium (Ti) or other metallic material which enable fusion and osseointegration of the implant in the disc space, including, among others, stainless steel, titanium alloys, nitinol, platinum, tungsten, silver, palladium, gold, cobalt chrome alloys, shape memory nitinol and mixtures thereof.

Additionally, the upper and lower implant sections 20, 30, 120, 130, 220 and 230 may have a porosity aspect in order to improve fixation of the implant 10, 110 and 210. The bone-contacting surfaces, serrations or teeth of the implant may have porosity of appropriate or desired sizes and geometry or configuration for optimal and rapid bony in growth. The bone-contacting surfaces, serrations or teeth porosity may have pores that are non-connected or interconnected pores with selected or desired pore size diameters, for example in the range between 1 to 1000 micrometers, preferably between 50 and 250 micrometers. The porosity may have predetermined patterns or have a porosity that has a random geometry or configuration in nature. The upper and lower implant sections 20, 30, 120, 130, 220 and 230 can be further coated or filled with osseoconductive and/or osseoinductive biomaterials such as hydroxyapatite (HA) and human recombinant bone morphogenic protein (rh BMP2). Those of skill in the art will recognize that the pore sizes, pore configuration, pore coating, and/or pore inter-connectivity aspect may be selected or vary for a particular spinal implant 10, 110 or 210 depending on needs or requirements of a physician, procedure or medical application. The spinal implant 10, 11, 210 can be made or manufactured by typical or known techniques and methods know to those of skill in the art, including among others, machining, molding, extrusion, stamping, laser processing, water-jet cutting or combination thereof.

The implant 10, 110 or 210 may be implanted in the disc space 101 using known methods, procedures and approaches, including a posterior (PLIF), direct lateral (DLIF), anterior (ALIF), translateral (TLIF) or any other suitable oblique direction and approach, as those of skill in the art will recognize. Further, a spinal implant may be delivered and inserted through known surgical technique and procedures, including: open, mini-open, minimal access spinal technologies (MAST) or other minimally invasive surgical (MIS) techniques.

In one approach, the implant 10, 110 or 210 is inserted via a direct lateral (DLIF) approach, for example as shown in FIG. 1. In one aspect, the implant 10, 110 or 210, shown in FIGS. 2-7B, will have a selected or desired physical shape and size for use in a spinal medical procedure. Those of skill in the art will readily recognize that the implant 10, 110 or 210 may take on any shaped desired or required for a particular medical use or application. Further, those of skill in the art will recognize that the implant may also be a dynamic vertebral implant device, with varying shape and size depending on the medical application where the implant used.

Prior to insertion, known medical instruments and tools may be used to prepare the intervertebral disc space 101, including pituitary rongeurs and curettes for reaching the nucleus pulposus or other area in the disc space 101. The disc space 101 may be prepared with a partial or complete discectomy. Ring curettes may be used as necessary to scrape abrasions from the vertebral endplates 103 and 107. Using such instruments, a location which will accept the collapsed implant 10, 110 or 210 is prepared in the disc space 101. Those of skill in the art will recognize that the collapsed implant 10, 110 or 210 may be positioned at any desired location between the adjacent vertebral bodies 103 and 107 depending on the surgeon's need and the performed surgical procedure or medical application.

The collapsed implant 10, 110, 210 is then inserted into the prepared disc space 101 using insertion instruments which are appropriate with the shape and configuration of the implant and surgical procedure to be used. A medical imaging technique and device may be used to visualize the implant 10, 110 or 210 during the insertion procedure by taking advantage of the implant's radiolucent and/or radiopaque properties. During the insertion step, the enhanced implant visualization will permit the surgeon to better maneuver and control the trajectory, position and orientation of the collapsed implant 10, 110 or 210 into the vertebral disc space 101 and through the surrounding patient anatomical environment.

The collapsed implant 10, 110 or 210 is then delivered into the intervertebral disc space 101 and positioned in a selected location and orientation between the end plates 103 and 107 of the adjacent vertebral bodies 100 and 105. The implant is inserted into the disc space 101 such that the implant upper and lower surface serrations or teeth are positioned between and adjacent to the upper and lower vertebral endplates 103 and 107. The implant's serrations may engage the vertebral endplates 103 and 107 to provide stability to the implant 10, 110 or 210. Once implanted, the implant is selectably and controbably expanded such that the implant's surface serrations or teeth will contact the upper and lower vertebral end plates 103 and 107 to form an interface between the implant 10, 110 or 210 and the adjacent vertebral bodies 100 and 105. The expanded implant will impart selected or desired height, lordotic or angular expansion, or a combination of height and lordotic or angular expansion. After the insertion and expansion of the implant 10, 110 or 210 between the vertebral bodies 100 and 105 has been completed, the implant graft material will promote the fusion or joining together of the vertebral bodies 100 and 105.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

While embodiments of the invention have been illustrated and described in detail in the present disclosure, the disclosure is to be considered as illustrative and not restrictive in character.

While embodiments of the invention have been illustrated and described in the present disclosure, the disclosure is to be considered as illustrative and not restrictive in character. The present invention may be carried out in other specific ways than those herein set forth without departing from the scope and essential characteristics of the invention. The present embodiments are, therefore, to be considered in all respects as illustrative and not restrictive, and all changes and modifications that come within the spirit of the invention are desired to be protected and are to be considered within the scope of the disclosure. Further, all changes coming within the meaning and equivalency range of the appended claims are also intended to be embraced therein.

1. A spinal implant for insertion into and positioning in an intervertebral disc space, the implant comprising:
a first implant section;
an opposing second implant section; and
at least one expander attached between the first and second implant sections;
wherein the at least one implant expander can be selectively expanded to thereby actuate the spinal implant to an expanded position.

2. The implant of claim 1, wherein the at least one implant expander is expanded via injection of an expanding material.

3. The implant of claim 1, wherein the at least one implant expander is selectively expanded to impart substantially vertical expansion to the spinal implant.

4. The implant of claim 1, wherein the at least one implant expander is selectively expanded to impart angular expansion to the spinal implant.

5. The implant of claim 1, wherein the at least one implant expander is selectively expanded to impart vertical and angular expansion to the spinal implant.

6. The implant of claim 1, wherein the spinal implant comprises
a first expander attached between the first and second implant sections; and
an opposing second expander attached between the first and second implant sections.

7. The implant of claim 2, wherein the expanding material is bone cement.

8. The implant of claim 1, wherein the at least one expander is a balloon.

9. The implant of claim 3, wherein the at least one implant expander imparts a substantially vertical expansion in the range of between eight and fourteen millimeters (8-14 mm).

10. The implant of claim 4, wherein the at least one implant expander imparts an angular expansion in the range of between zero degrees to twelve degrees (0°-12°.

11. The implant of claim 1, wherein the first and second implant sections are polyetheretherketone (PEEK) or a metallic material.

12. The implant of claim 11, wherein the metallic material is titanium (Ti) or a titanium (Ti) alloy.

13. The implant of claim 1, wherein the first and second implant sections comprise a Hydroxyapatite (HA) layer.

14. A spinal implant for insertion into and positioning in an intervertebral disc space, the implant comprising:
a first implant section comprising bone securing serrations;
an opposing second implant section comprising bone securing serrations;
a first lateral expander attached between the first and second implant sections; and
an opposing second lateral expander attached between the first and second implant sections.
wherein the first and second lateral expanders can be selectively expanded to thereby actuate the spinal implant to an expanded position.

15. The implant of claim 14, wherein the first and second lateral expanders are expanded via injection of an expanding material.

16. The implant of claim 14, wherein the first and second lateral expanders are selectively expanded to impart substantially vertical expansion to the spinal implant.

17. The implant of claim 1, wherein the first and second lateral expanders are selectively expanded to impart angular expansion to the spinal implant.

18. The implant of claim 1, wherein the first and second lateral expanders are selectively expanded to impart vertical and angular expansion to the spinal implant.

19. The implant of claim 1, wherein the first and second implant sections are polyetheretherketone (PEEK).

20. The implant of claim 1, wherein the first and second implant sections comprise a Hydroxyapatite (HA) layer.
